(19) United States
(12) Patent Application Publication
(10) Pub. No.: US 2012/0322824 A1
(43) Pub. Date: Dec. 20, 2012

(54) Cocaine Antagonist/Antidepressant Pharmaceutical Preparations

(76) Inventors: Christopher K. Surratt, Pittsburgh, PA (US); Jeffry D. Madura, Pittsburgh, PA (US); Martin Indarte, Buenosaires (AR); David Lapinsky, Allison Park, PA (US); Jeffery Talbot, Bluffton, OH (US)

(21) Appl. No.: 13/325,618

(22) Filed: Dec. 14, 2011

Related U.S. Application Data
(60) Provisional application No. 61/460,557, filed on Jan. 4, 2011.

(51) Int. Cl. A61K 31/445; A61P 25/30; A61P 25/24

(52) U.S. Cl. 514/317

(57) ABSTRACT

The present invention is a psychostimulant antagonist or antidepressant in pharmaceutical unit dosage form—with each unit dosage form containing in an effective amount to treat cocaine or amphetamine dependence in an animal or human in which such treatment is indicated and to whom one or more such unit dosage forms are administered—comprising one or more substituted or unsubstituted diphenyl piperidine derivative active agents according to Formula I:

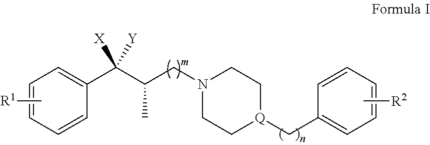

including a diastereomer or enantiomer thereof, together with one or more pharmaceutically acceptable excipients or diluents.

### CROSS-REFERENCE TO RELATED APPLICATIONS

This patent application claims priority to, and incorporates by reference, U.S. Provisional Patent Application Ser. No. 61/460,557 filed 4 Jan. 2011.

### STATEMENT OF FEDERALLY SPONSORED RESEARCH OR DEVELOPMENT

The subject matter of this patent application was developed at least in part under National Institute on Drug Abuse, National Institutes of Health, Contract No. 5R01DA026530; the U.S. Government has certain rights to this invention.

### BACKGROUND OF THE INVENTION

1. Field of the Invention

The invention pertains to an active agent, and pharmaceutical composition containing it, which is useful as an antagonist to treat addiction to or dependence on cocaine or other psychostimulant substances of abuse.

2. Description of Related Art

Unlike the treatment of opioid or alcohol addiction or dependence, prior to the present innovation there were no known suitable medications available in the United States or international pharmaceutical markets to treat addiction to or dependence on abused psychostimulant drugs such as cocaine or amphetamines. Naltrexone, a substituted oxymorphone, is both an opioid antagonist and an ethanol antagonist and is well known for use in treating both alcohol or opioid dependence or addiction, sometimes in implant form or rapid detoxification settings in addition to other routes of administration. Opioid/alcohol antagonists also include naloxone and naloxazone, without limitation. Despite the existence of multiple drugs that can block or antagonize opioids or alcohol, previous searches for drugs antagonistic to cocaine or other stimulants have heretofore proved fruitless.

Recognition of psychostimulants such as cocaine and the amphetamines by the dopamine transporter (DAT) protein is principally responsible for the euphoria and addiction associated with these drugs. In investigation of possible cocaine antagonists, the challenge to date has not been that cocaine antagonist candidates do not already exist, but that the compounds or drugs found to mimic DAT inhibition also mimic the euphoria or other perceived rewards of cocaine. Prior to the present invention, therefore, drugs or compounds which could serve as cocaine antagonists themselves had unacceptably high potential for abuse as psychostimulant substances. A need thus remains for a cocaine antagonist which can effectively mimic the DAT inhibition of cocaine without creating euphoria or reward in the patient thus treated, to serve as a pharmaceutical treatment in support of patients recovering from psychostimulant dependence or addiction.

### SUMMARY OF THE INVENTION

The present invention is a psychostimulant antagonist in pharmaceutical unit dosage form—with each unit dosage form containing in an effective amount to treat cocaine or amphetamine dependence in an animal or human in which such treatment is indicated and to whom one or more such unit dosage forms are administered—comprising one or more diphenyl piperidine derivative active agents according to Formula I

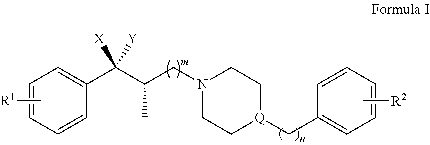

wherein
1. R¹ is —H, 4—OH, 4—Cl, 4—OMe, 3—Cl, 4—NMe₂, 4—NH₂, 3—Me—4—NMe₂, 4—Me, 4-t-Bu, 3—Cl, 3—NMe₂, 3—Me, 3—CF₃, 3—Br, 3—I, 2—Cl, 3,5—Cl₂, 4—NO₂, 3—NO₂, 4—F, 3,4—Cl₂, 3—CF₃-4—Cl, 4—CF₃, 4—Br, 4—I, 3—CF₃-4—NO₂, 2,4—Cl₂, 4—OH—3—OH, 4—OH—3—SH or 4—SH—3—OH;
2. R² is —H, 4—OH, 4—Cl, 4—OMe, 3—Cl, 4—NMe₂, 4—NH₂, 3—Me—4—NMe₂, 4—Me, 4-t-Bu, 3—Cl, 3—NMe₂, 3—Me, 3—CF₃, 3—Br, 3—I, 2—Cl, 3,5—Cl₂, 4—NO₂, 3—NO₂, 4—F, 3,4—Cl₂, 3—CF₃-4—Cl, 4—CF₃, 4—Br, 4—I, 3—CF₃-4—NO₂, 2,4—Cl₂, 4—OH—3—OH, 4—OH—3—SH or 4—SH—3—OH;
3. X is —H;
4. Y is —OH or —H;
5. or both X and Y taken together are —O;
6. Q is —CH or —N;
7. m is an integer from 1 to 4; and
8. n is an integer from 1 to 4,
   or a diastereomer or enantiomer thereof, together with one or more pharmaceutically acceptable excipients or diluents.

### BRIEF DESCRIPTION OF THE DRAWING(S)

FIGS. 1a and 1b are bar graphs showing the results of “conditioned place preference” testing of a diphenyl piperidine derivative according to the invention, in mice;

FIG. 2a-d are bar graphs showing the results of “locomotor activity” testing of a diphenyl piperidine derivative according to the invention, in mice;

FIG. 3 is a bar graph showing the results of an “open field test” of a diphenyl piperidine derivative according to the invention, in mice;

FIG. 4a-d are bar graphs showing the results of a second set of “locomotor activity” tests of a contrasting active agent MI-17, in mice; and

FIG. 5a-d are bar graphs showing the results of “tail suspension” testing (TST) and “forced swim” testing (FST) of a diphenyl piperidine derivative according to the invention, in mice.

### DETAILED DESCRIPTION OF THE INVENTION

As described above, the present invention is a psychostimulant antagonist in pharmaceutical unit dosage form—with each unit dosage form containing in an effective amount to treat cocaine or amphetamine dependence in an animal or human in which such treatment is indicated and to whom one or more such unit dosage forms are administered—comprising one or more diphenyl piperidine derivative active agents according to Formula I

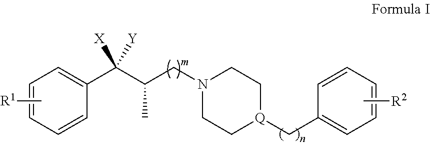

wherein
1. R¹ is —H, 4—OH, 4—Cl, 4—OMe, 3—Cl, 4—NMe₂, 4—NH₂, 3—Me—4—NMe₂, 4—Me, 4t-Bu, 3—Cl, 3—NMe₂, 3—Me, 3—CF₃, 3—Br, 3—I, 2—Cl, 3,5—Cl₂, 4—NO₂, 3—NO₂, 4—F, 3,4—Cl₂, 3—CF₃-4—Cl, 4—CF₃, 4—Br, 4—I, 3—CF₃-4—NO₂, 2,4—Cl₂, 4—OH—3—OH, 4—OH—3—SH or 4—SH—3—OH;
2. R² is —H, 4—OH, 4—Cl, 4—OMe, 3—Cl, 4—NMe₂, 4—NH₂, 3—Me—4—NMe₂, 4—Me, 4-t-Bu, 3—Cl, 3—NMe₂, 3—Me, 3—CF₃, 3—Br, 3—I, 2—Cl, 3,5—Cl₂, 4—NO₂, 3—NO₂, 4—F, 3,4—Cl₂, 3—CF₃-4—Cl, 4—CF₃, 4—Br, 4—I, 3—CF₃-4—NO₂, 2,4—Cl₂, 4—OH—3—OH, 4—OH—3—SH or 4—SH—3—OH;
3. X is —H;
4. Y is —OH or —H;
5. or both X and Y taken together are —O;
6. Q is —CH or —N;
7. m is an integer from 1 to 4; and
8. n is an integer from 1 to 4,
   or a diastereomer or enantiomer thereof, together with one or more pharmaceutically acceptable excipients or diluents.

The active agents of the present invention may be formulated for administration by any of various routes. The active agents may include an excipient in combination one or more active agents, and may be in the form of, for example, tablets, capsules, powders, granules, lozenges, pill, suppositories, liquid or gel preparations. Alternatively, one or more active agents may be formulated for parenteral administration in a sterile medium. The active agent may be formulated for a subdermal implant in the form of a pellet, rod or granule. The implant or implants may be inserted subcutaneously by open surgery or by use of a trochar and cannula or any other implant device or technique under local anaesthesia. The implant may be periodically replaced or removed altogether. One or more active agents may also be formulated for transdermal administration using a patch. The patch is applied to an optionally shaven area of the skin of the patient while the one or more active agents are desired for administration, and removed when no longer needed. Conventional pharmaceutical practice may be employed to provide suitable formulations or compositions to administer the active agents to patients suffering from or presymptomatic for cocaine dependence or addiction. The active agents may be administered systemically or may be administered directly to the central nervous system (CNS). In some embodiments, active agents according to the invention may be provided in a form suitable for delivery across the blood brain barrier. Any appropriate route of administration may be employed, for example, parenteral, intravenous, subcutaneous, intramuscular, intracranial, intraorbital, ophthalmic, intraventricular, intracapsular, intraspinal, intracisternal, intraperitoneal, intranasal, aerosol, or oral administration. Therapeutic formulations may be in the form of liquid solutions or suspensions because the active agent(s) may be either dissolved or suspended in the accompanying excipient or diluent medium; for oral administration, formulations may be in the form of tablets or capsules; and for intranasal formulations, the formulations may be in the form of powders, nasal drops, or aerosols.

Methods well known in the art for making formulations are found in, as a single nonlimiting example, “Remington's Pharmaceutical Sciences” (19th edition), ed. A. Gennaro, 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, as nonlimiting examples, polyoxyethylene-9-lauryl ether, glycocholate and deoxycholate, or may be oily solutions for administration in the form of nasal drops, or as a gel. A pharmaceutically acceptable excipient includes any and all solvents, dispersion media, coatings, antibacterial, antimicrobial or antifungal agents, isotonic and absorption delaying agents, and the like that are physiologically compatible. The excipient may be suitable for intravenous, intraperitoneal, intramuscular, intrathecal or oral administration. The excipient may include sterile aqueous solutions or dispersions for extemporaneous preparation of sterile injectable solutions or dispersion. Use of such media for preparation of medicaments is known in the art.

For therapeutic or prophylactic compositions, the compounds are administered to an individual in an amount sufficient to create partial or complete DAT inhibition. An “effective amount” of a compound according to the invention includes a therapeutically effective amount or a prophylactically effective amount. A “therapeutically effective amount” refers to an amount effective, at dosages and for periods of time necessary, to achieve the desired therapeutic result, such as reduction of dependence or addiction to cocaine or other psychostimulant substances of abuse. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A preferred range for therapeutically effective amounts of an active agent according to the invention may be 0.1 nM-0.1M, 0.1 nM-0.05M, 0.05 nM-15 micromolar or 0.01 to 10 micromolar. A pharmaceutically effective amount of an active agent according to the invention is the same as a therapeutically effective amount. The actual amount which constitutes a therapeutically or pharmaceutically effective amount may be dependent on mode of delivery, time period of the dosage, age, weight, general health, sex and diet of the subject receiving the medicament.

Dosage values may vary with the severity of the condition to be alleviated or with the route of administration selected. For example, for oral administration, dosage values may be higher than for intravenous or intraperitoneal administration. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, active agents and preparations of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

Active agents of the invention can be provided alone or in combination with other compounds (for example, nucleic acid molecules, small molecules, peptides, or peptide analogues), in the presence of a liposome, an adjuvant, or any pharmaceutically acceptable carrier, in a form suitable for administration to humans. If desired, treatment with one or more active agents or preparations according to the invention may be combined with other therapies for substance dependence or addiction, including but not limited to nonsteroidal antiinflammatory or other benign analgesics or other concomitant therapies such as anxiolytic agents or preparations or other adjuvant therapies. Psychotherapy may be an adjunct treating aid in combination with the present active agent therapy compositions and methods.

### EXAMPLES

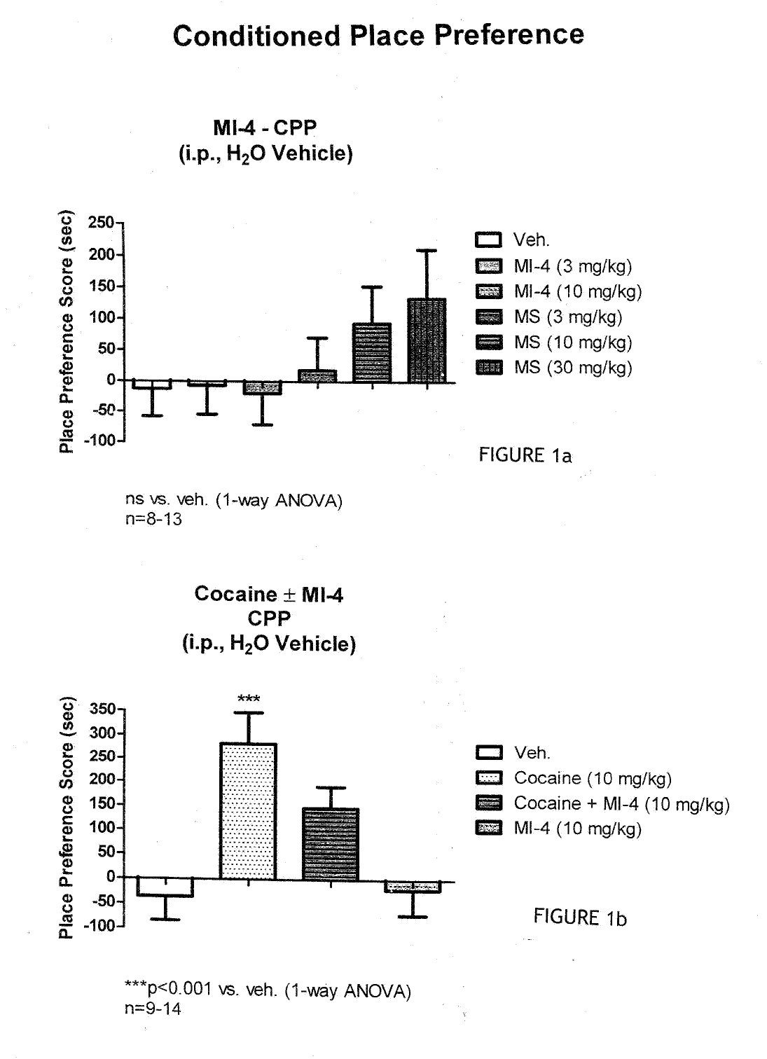

RO-25-6981 (referred to below as MI-4) was tested for the ability to antagonize or block the rewarding effects of cocaine in rodents. RO-25-6981 is a diphenyl piperidine derivative within the scope of Formula I and specifically is RO-25-6981 hydrochloride or ([R-(R*,S*)]-α-(4-Hydroxyphenyl)-β-methyl-4-(phenylmethyl)-1-piperidinepropanol such as is disclosed at paragraph 11 of U.S. Published Patent Application No. 2010/0048653 as an NMDA receptor modulator. Alternative nomenclature is ([R—(R)]-α-(4-Hydroxyphenyl)-β-methyl-4-(phenylmethyl)-1-piperidinepropanol. In order to understand the present invention, it is important to recognize that there are many known NMDA receptor modulators (that is, N-methyl D-Aspartate receptor modulators, with N-methyl D-Aspartate receptor's being one of the known glutamate receptors known in the field of neuroprotective modulation) that do not function as cocaine antagonists such that there is no tacit suggestion in 2010/0048653 of any possibility of the present invention. The studies reported in the following examples exploit similarities between rodents and humans wherein individuals show preference for environments associated with receiving an addictive substance, such as cocaine. The results of the tests reported in these Examples are shown in FIGS. 1-5.

### Example 1

The conditioned place preference assay (CPP) is one test commonly used to show the ability of an antagonist to block rewarding effects in mice. Referring now to FIGS. 1a-b, according to the CPP test mice are placed in an apparatus with two chambers that differ in both visual and tactile cues. Over the course of a one-week training period, a rewarding drug (e.g.—cocaine) is administered once daily exclusively in one chamber so as to allow animals to associate the cues of that chamber with the drug. Sterile water is administered once daily while the animals occupy the opposite chamber to control for non-drug effects. At the conclusion of the training period, animals are allowed free access to both chambers and the time spent in each chamber is measured electronically. It is well known that addictive substances increase the amount of time animals spend in the drug-paired chamber, in the absence of any intervention.

Using the CPP test, the animals receiving cocaine (10 mg/kg) via intraperitoneal (i.p.) injection spent significantly more time in the drug-paired chamber compared to animals injected with sterile water alone. However, when MI-4 (10/mg/kg, i.p.) was co-administered with cocaine, the effect of the cocaine was reduced by half (referring to the central two data bars in FIG. 1b). Importantly, MI-4 did not exhibit rewarding effects when administered alone at the same dose (e.g.—no increase was observed in the time spent in the MI-4-paired chamber). Moreover, additional testing was performed to control for effects of MI-4 on general behavioral activity, an essential consideration when interpreting results from locomotor-based behavioral paradigms such as the CPP test.

### Example 2

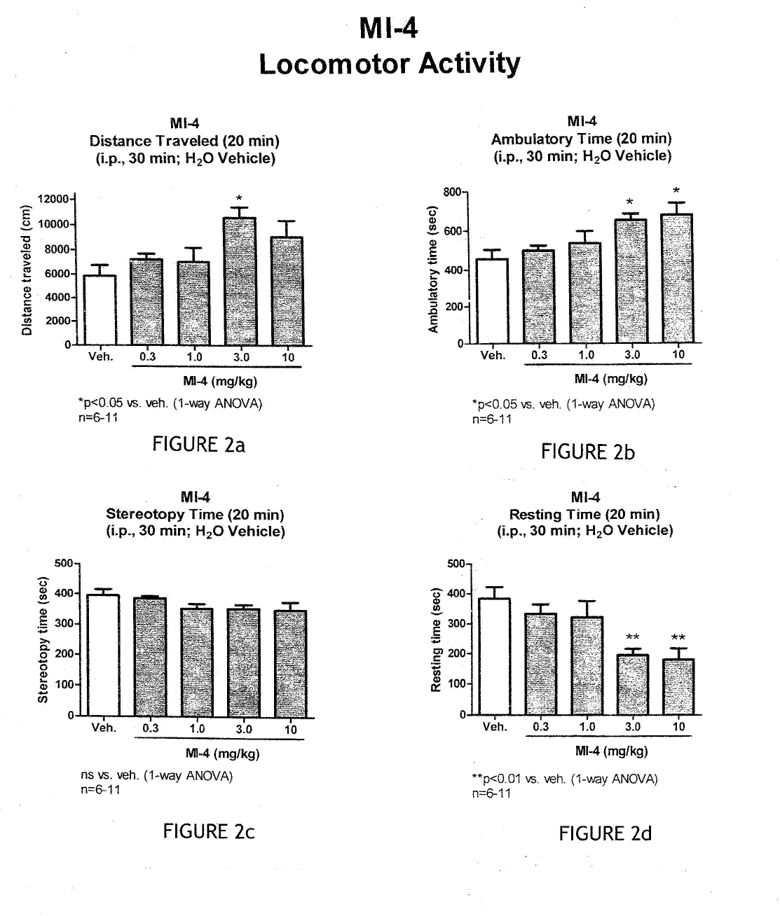

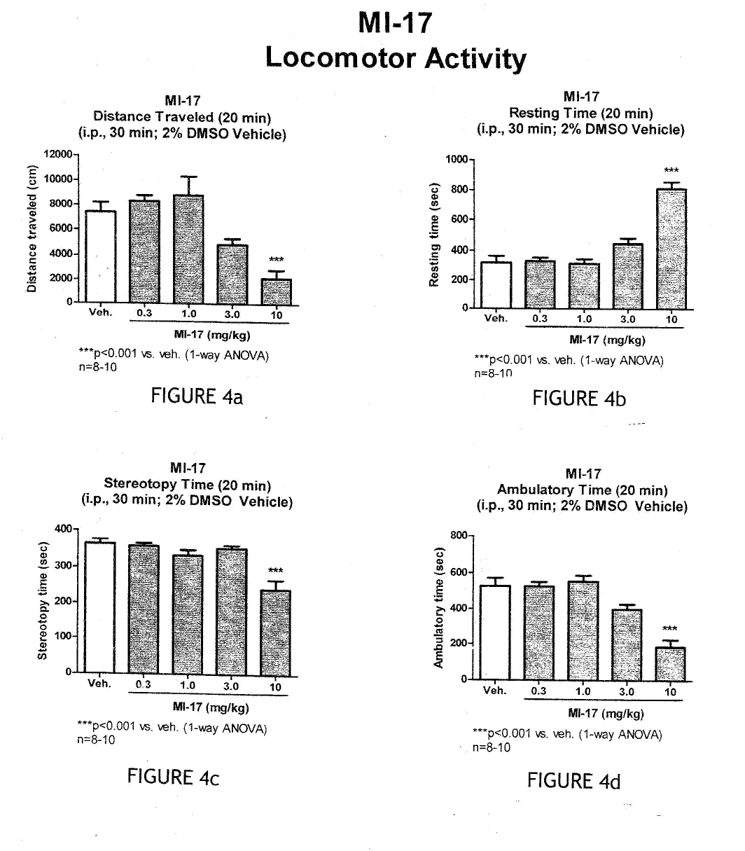

Referring now to FIGS. 2a-d, locomotor activity was assessed in a non-invasive manner using a Opto-Varimax 4 Activity Analyzer (Columbus Instruments, Columbus, Ohio) that measures animal activity in a cage intersected with photocells projecting infrared beams 2.5 cm apart and 2 cm above the floor. After a brief habituation period, animals were treated with either sterile water as vehicle or MI-4 and activity was measured for 20 min. In testing a compound according to the present invention, MI-4, and also a compound not falling within Formula I, namely, MI-17 pictured below, we found that MI-4 had only minor effects on the locomotor activity profile, which includes the distance traveled by animals during the treatment period measured in cm (FIG. 2a), the ambulatory time or the amount of time animals were mobile measured in sec (FIG. 2b), and the resting time or the amount of time animals spent at rest measured in sec (FIG. 2d). In addition, MI-4 did not significantly alter stereotopy time or the amount of time animals spent exhibiting grooming behavior during the treatment period (FIG. 2c). These data largely confirm findings from other laboratories that MI-4 only modestly increases activity with little or no effect on gross behaviors, and suggests that based on the CPP test results, MI-4 appears to interfere with the euphoric or rewarding properties of cocaine. In contrast, as shown in FIGS. 4a-d, MI-17 shown below

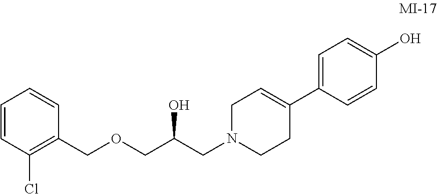

significantly altered spontaneous activity and gross behaviors. Beginning at 3 mg/kg but mostly at 10 mg/kg, MI-17 dramatically decreased the distance traveled (FIG. 4a), stereotopy time (FIG. 4c), and ambulatory time (FIG. 4d) of treated animals while dramatically increasing their observed resting time (FIG. 4b). These data suggest MI-17 has potentially debilitating effects on animal activity and gross behavior, and that its use in behavioral-based paradigms at doses higher than 1 mg/kg, i.p. is contraindicated.

### Example 3

In addition to its effects as a putative cocaine antagonist, another therapeutic consideration of MI-4 and analogs is their potential use as antidepressants. Current antidepressant therapies are largely based on a similar mechanism of improving monoamine neurotransmitter function by inhibiting their reuptake from the synaptic cleft. Indeed, the most widely prescribed antidepressants target individually or in combination the serotonin (5-HT), dopamine (DA), or noreinephrine (NE) transporters. Because in vitro studies have shown that MI-4 exhibits physiologically-relevant binding affinity for each of these transporters, we investigated whether MI-4 would exhibit antidepressant or anxiolytic properties in vivo.

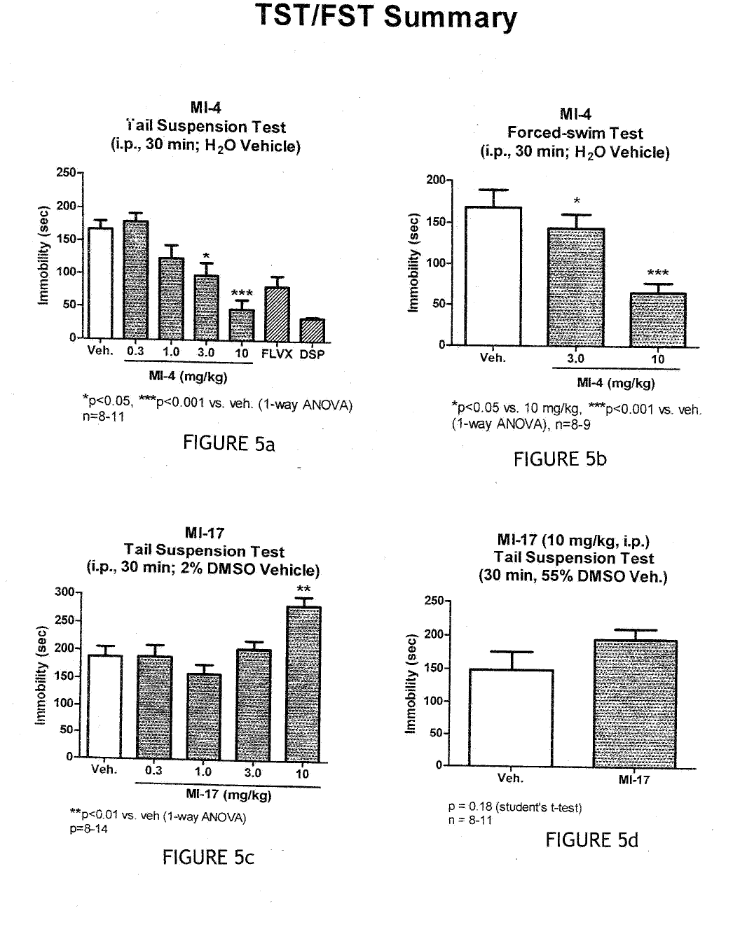

Referring now to FIGS. 5a-d, the tail suspension test (TST) and forced swim test (FST) are well-established, prototypical rodent models of learned helplessness. They are strongly correlated to drugs that possess antidepressant-like activity in humans. These assays are based on the premise that helpless or “depressed” animals struggle less to escape the mild but inescapable stress of being suspended or being immersed in room temperature water. This is based in part on established findings that drugs that possess antidepressant activity in humans reduce the immobility time of the animal in these tests. In the TST, standard laboratory tape is used to immobilize an animal by its tail to a fixed support approximately 10.5 inches above a surface. The duration of the test is six minutes during which time animal movement is videotaped and later scored for immobility time (in sec) by a blinded observer. Similarly, in the FST animals are immersed in approximately 10 cm of 25° C. water in a 20 cm×13 cm glass cylinder. Animals were tested for 5 min and behavior was videotaped and scored for immobility time (sec) by a blinded observer. We found that animals treated with MI-4 produced a dose-dependent reduction in immobility time in the TST that was comparable in effect to the serotonin selective reuptake inhibitor fluovoxamine and the norepinephrine selective reuptake inhibitor desipramine (FIG. 5a). Similar findings were obtained when MI-4 was tested in the FST (FIG. 5b). These data suggest that MI-4 has both potent and efficacious antidepressant activity. In contrast, MI-17 dose-dependently increased immobility times in the TST in two different preparations: 2% DMSO vehicle (FIG. 5c) or 55% DMSO vehicle in the oxalate form (FIG. 5d). Based on the locomotor activity data previously presented (FIG. 4a-d), it is likely that these effects of MI-17 in the TST result from its ability to profoundly inhibit spontaneous locomotor activity.

### Example 4

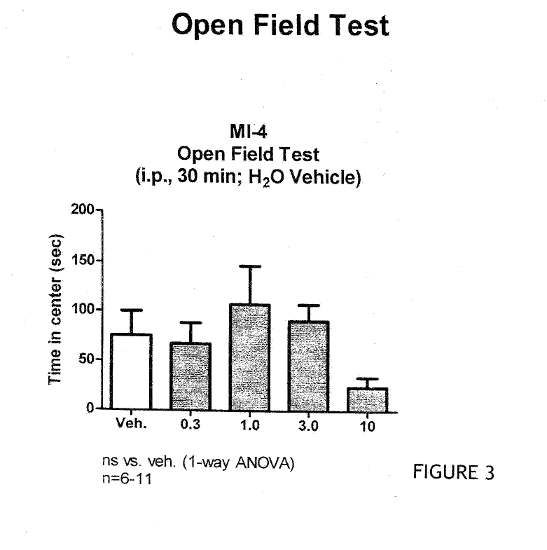

Anxiety disorders are often co-morbid with depression and antidepressant drugs are used in the treatment of anxiety. Tests for anxiety in rodents, such as the open field test, make use of the aversion of the animals for exposed locations. Treatment with anxiolytics such as diazepam or fluoxetine increases the amount of time an animal will spend in the center of the open field. We used the Opto-Varimax 4 Activity Analyzer to assess the time animals spent in the center of the open field vs. the periphery after treatment with MI-4. Data were measured as time spent in center (TIC). Referring now to FIG. 3, we found that MI-4 did not significantly increase the time spent in center in the open field. These data suggest that MI-4 does not have a strong anxiolytic effect in the open field test, with the caveat that the decreased time in center at 10 mg/kg is likely a locomotor rather than anxiolytic effect. However, on their own, these data do not rule out MI-4 anxiolytic action which is otherwise apparent.

Active agents within Formula I are useful as therapy for the toxicity and/or abuse of cocaine and as antidepressants. Up until the present invention there has been some debate whether a class of active agents could ever be developed that blocked DAT binding without also blocking dopamine uptake, because the prediction was that a compound that blocked both DAT binding and dopamine uptake would also be likely to be abused as a psychostimulant itself In theory, without desire of being bound by the theory, the active agents of Formula I as defined are all able to block DAT binding without blocking dopamine uptake. Moreover, human studies involving traxoprodil, an ifenprodil analog closely related to MI-4, provide insight into the pharmacokinetic characteristics of the class of NR2B antagonists, such as MI-4. In human subjects, sustained levels of traxoprodil both in the plasma and cerebral spinal fluid (>200 ng/ml) are achieved with intravenous administration for up to 72 hours with no observable adverse effects. Traxoprodil also exhibits extensive oral bioavailability in human subjects across a spectrum of poor metabolizers (PM; approx. 80%) and extensive metabolizers (EM; 23-62%). Numerous additional studies using animal models show that non-intravenous routes of administration are viable for MI-4 delivery to target tissues, including the central nervous system, and that systemic distribution and metabolism of MI-4 is suitable for therapeutic dosing. In addition, these studies indicate that MI-4 is well tolerated in rodents with no observable adverse effects at doses less than 100 mg/kg, i.p. These findings point to MI-4 and MI-4 analogs as therapeutic agents in which the oral route of administration is feasible.

Although the invention has been described with particularity above, with specific reference to methods and compositions and their administration as pharmaceutically active agents, the invention is only to be limited insofar as is set forth in the accompanying claims.

1. A psychostimulant antagonist/antidepressant pharmaceutical composition in unit dosage form, comprising one or more diphenyl piperidine derivative active agents according to Formula I 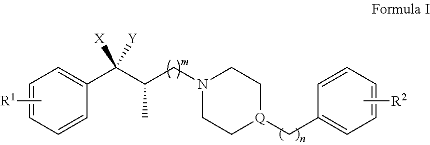
wherein
1. R¹ is —H, 4—OH, 4—Cl, 4—OMe, 3—Cl, 4—NMe₂, 4—NH₂, 3—Me—4—NMe₂, 4—Me, 4-t-Bu, 3—Cl, 3—NMe₂, 3—Me, 3—CF₃, 3—Br, 3—I, 2—Cl, 3,5—Cl₂, 4—NO₂, 3—NO₂, 4—F, 3,4—Cl₂, 3—CF₃-4—Cl, 4—CF₃, 4—Br, 4—I, 3—CF₃-4—NO₂, 2,4—Cl₂, 4—OH—3—OH, 4—OH—3—SH or 4—SH—3—OH;
2. R² is —H, 4—OH, 4—Cl, 4—OMe, 3—Cl, 4—NMe₂, 4—NH₂, 3—Me—4—NMe₂, 4—Me, 4-t-Bu, 3—Cl, 3—NMe₂, 3—Me, 3—CF₃, 3—Br, 3—I, 2—Cl, 3,5—Cl₂, 4—NO₂, 3—NO₂, 4—F, 3,4—Cl₂, 3—CF₃-4—Cl, 4—CF₃, 4—Br, 4—I, 3—CF₃-4—NO₂, 2,4—Cl₂, 4—OH—3—OH, 4—OH—3—SH or 4—SH—3—OH;
3. X is —H;
4. Y is —OH or —H;
5. or both X and Y taken together are —O;
6. Q is —CH or —N;
7. m is an integer from 1 to 4; and
8. n is an integer from 1 to 4,
or a diastereomer or enantiomer thereof, together with one or more pharmaceutically acceptable excipients or diluents;
wherein each unit dosage form contains an amount of one or more diphenyl piperidine active agents effective to treat psychostimulant dependence or addiction in an animal or human in need of treatment for psychostimulant dependence or addiction when one or more of such unit dosage forms are administered to said animal or human.

2. A method for treating an animal or human in need of treatment for depression or for psychostimulant dependence or addiction, comprising administering one or more dosages of a pharmaceutical composition in unit dosage form comprising: an effective amount of one or more diphenyl piperidine derivative active agents according to Formula I 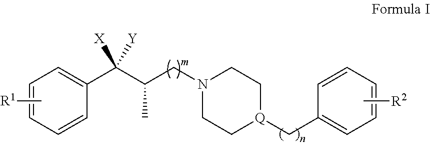
wherein
1. R¹ is —H, 4—OH, 4—Cl, 4—OMe, 3—Cl, 4—NMe₂, 4—NH₂, 3—Me—4—NMe₂, 4—Me, 4-t-Bu, 3—Cl, 3—NMe₂, 3—Me, 3—CF₃, 3—Br, 3—I, 2—Cl, 3,5—Cl₂, 4—NO₂, 3—NO₂, 4—F, 3,4—Cl₂, 3—CF₃-4—Cl, 4—CF₃, 4—Br, 4—I, 3—CF₃-4—NO₂, 2,4—Cl₂, 4—OH—3—OH, 4—OH—3—SH or 4—SH—3—OH
2. R² is —H, 4—OH, 4—Cl, 4—OMe, 3—Cl, 4—NMe₂, 4—NH₂, 3—Me—4—NMe₂, 4—Me, 4-t-Bu, 3—Cl, 3—NMe₂, 3—Me, 3—CF₃, 3—Br, 3—I, 2—Cl, 3,5—Cl₂, 4—NO₂, 3—NO₂, 4—F, 3,4—Cl₂, 3—CF₃-4—Cl, 4—CF₃, 4—Br, 4—I, 3—CF₃-4—NO₂, 2,4—Cl₂, 4—OH—3—OH, 4—OH—3—SH or 4—SH—3—OH
3. X is —H;
4. Y is —OH or —H;
5. or both X and Y taken together are —O;
6. Q is —CH or —N;
7. m is an integer from 1 to 4; and
8. n is an integer from 1 to 4,
or a diastereomer or enantiomer thereof, together with one or more pharmaceutically acceptable excipients or diluents;
wherein each unit dosage form contains an amount of one or more diphenyl piperidine active agents effective to treat psychostimulant dependence or addiction in an animal or human in need of treatment for psychostimulant dependence or addiction when one or more of such unit dosage forms are administered to said animal or human.
